(19) United States
(12) Patent Application Publication
(10) Pub. No.: US 2009/0280062 A1
(43) Pub. Date: Nov. 12, 2009

(54) LONGEVITY-PROMOTING EFFECTS OF ACETIC ACID AND REISHI POLYSACCHARIDE

(76) Inventors: Chi-Huey Wong, La Jolla, CA (US); Ming-Hong Chuang, Taipei County (TW); Wen-Bin Yang, Taipei County (TW); Shyh-Horng Chiou, Kaohsiong (TW)

Correspondence Address: Luce, Forward, Hamilton & Scripps LLP, 2050 Main Street, Suite 600, Irvine, CA, 92614, US

(21) Appl. No.: 12/425,319

(22) Filed: Apr. 16, 2009

Related U.S. Application Data
(60) Provisional application No. 61/045,911, filed on Apr. 17, 2008.

(51) Int. Cl. A61K 49/00; A61K 31/715; C12Q 1/68; A61K 31/19; A61P 39/00

(52) U.S. Cl. 424/9.2; 514/54; 435/6; 514/557

(57) ABSTRACT

A composition of acetic acid and a composition of acetic acid and RF₃ effecting expression of DAF-16 in C. elegans, wherein the at least acetic acid and the RF₃ polysaccharide is effective to increase the life-span of C. elegans or humans. A method of regulating DAF-16 expression, comprising: administering a composition comprising at least acetic acid and RF₃ polysaccharide; providing the composition to at least one receptor on a surface of a cell; causing an increase in expression of DAF-16. A method for screening a series of compounds or constituents parts of compounds including some antioxidant vitamins, traditional herb medicines, and vinegars using C. elegans as a live organism model to examine to determine if they have an impact the lifespan of C. elegans and the underlying mechanism(s) thereof.

### RELATED APPLICATIONS

This application claims priority to U.S. provisional patent application Ser. No. 61/045,911, filed on Apr. 17, 2008, which is hereby incorporated by reference, as if fully set forth herein, and this application incorporates by reference, as if fully set forth herein, U.S. patent application Ser. No. 11/534,204, filed on Sep. 21, 2007 and published as U.S. patent publication no. 2007/0105814; and U.S. patent application Ser. No. 11/549,215, filed on Oct. 13, 2006 and published as U.S. patent publication no. 2007/0231339.

### FIELD OF DISCLOSURE

This disclosure relates to compounds and methods for the elevated gene expression of DAF-16/FOXO and screening systems for a potential to increase longevity in higher organisms and identification of compounds that exhibit such potential, including but not limited to elevated gene expression of DAF-16/FOXO.

### BACKGROUND

Aging is a fundamental process with great innate diversity. Aging, like many other biological processes, is subject to regulation by pathways that have been conserved during evolution. Changing single genes within these pathways can extend lifespan dramatically, causing an experimental animal to age normally but more slowly than usual. Some of these long-lived mutants live with extraordinary lifespan. In aging studies, two popular invertebrate model organisms were extensively used, i.e. Caenorhabditis elegans and Drosophila melanogaster.

C. elegans is especially a much-researched nematode in recent years. It is a small worm, just 1 mm in length that lives in the soil of temperate regions, where it feeds on bacteria. It has been used extensively for aging studies in part because of its short and consistent lifespan (average 14˜20 days at 20° C.). The lifespan-control mechanisms of C. elegans have been shown to be associated with the conserved insulin/IGF-1 DAF-2 signaling pathway. This insulin-signaling pathway includes the DAF-2 trans-membrane receptor, a series of intracellular kinases and the DAF-16 protein, which ultimately functions to both positively and negatively regulate the transcription of metabolic, chaperone, cellular defense, and other genes. The DAF-16 protein in regulation of the expression of various antioxidant enzymes e.g. superoxide dismutase (SOD) plays a major role in the regulation of the lifespan of C. elegans. The regulation of lifespan through this insulin-like signaling cascade is an evolutionarily conserved mechanism and has also been demonstrated to function in flies and mice. However, DAF-16 activity has also been shown to be modulated by the JNK signaling pathway, the SIR-2.1 deacetylase, HSF-1, LIN-14, and SMK-1 in the nucleus.

### SUMMARY

Previous research has shown that polysaccharides fraction 3 (RF₃) from Ganoderma lucidum possess an immuno-modulating effect through Toll-like receptor 4 (TLR4). When fed with the polysaccharide RF₃, tumor-implanted mice enjoyed prolonged lifespan, presumably due to the activation of a host immune response. Whether there is any ortholog of TLR in C. elegans and what its function might be is yet undetermined. However, a Toll-like receptor intracellular domain (TIR-1) has been clearly demonstrated to exist in this primitive worm. TIR-1 has also been shown to be associated with an antibacterial pathway in C. elegans. Therefore, RF₃ is a candidate to have a beneficial effect on the lifespan of C. elegans.

According to implementations of the present disclosure, a composition is disclosed, comprising, in combination: at least acetic acid and/or an RF₃ polysaccharide; wherein the at least acetic acid and/or the RF₃ polysaccharide is administered to regulate DAF-16 expression, wherein DAF-16 may have a sequence corresponding to SEQ ID NO:19. The composition may comprise between about 50 ppm to about 100 ppm acetic acid and between about 100 ppm and about 500 ppm RF₃ polysaccharide. The composition may further comprise at least one of an extract of Antrodia camphorate and an extract of Hericium erinaceus.

According to implementations of the present disclosure, a method of regulating DAF-16 expression is disclosed, comprising: administering a composition comprising at least acetic acid and/or an RF₃ polysaccharide; providing the composition to at least one receptor on a surface of a cell; causing an increase in expression of DAF-16, wherein DAF-16 may have a sequence corresponding to SEQ ID NO:19. The at least one receptor may include TLR4. The causing an increase in expression of DAF-16 may include activating the mitogen-activated protein kinase (MAPK) pathway, causing an increase in expression of RAB-1, causing an increase in expression of PMK-1, or inhibiting expression of DAF-2.

According to implementations of the present disclosure, a method of screening compounds for their effect on DAF-16 expression in C. elegans is disclosed, comprising: administering at least one known compound to C. elegans; comparing the lifespan of C. elegans to which the at least one known compound was administered with the lifespan of control C. elegans to which the at least one known compound was not administered; determining if the lifespan of C. elegans to which the at least one known compound was administered exceeded the normal lifespan by a selected percentage; and, determining if the at least one known compound increased at least DAF-16 expression in C. elegans. The at least one known compound may include acetic acid. Determining if the at least one known compound increased at least DAF-16 expression in C. elegans may be accomplished by performing reverse transcription of an isolated and purified RNA sample from the C. elegans to which the at least one known compound was administered. The isolated and purified RNA sample may include at least one of DAF-2, DAF-16, TIR-1, RAB-1 and PMK-1.

### DRAWINGS

The above-mentioned features and objects of the present disclosure will become more apparent with reference to the following description taken in conjunction with the accompanying drawings wherein like reference numerals denote like elements and in which:

FIGS. 1A-C are graphs which show the effect of test substances on the lifespan of C. elegans. The survival curves shown represent various sets of worms analyzed after treatment with the indicated substances. (A) Worms treated with water (control), acetic acid (50 ppm) and various vitamins (50 ppm). (B) Worms treated with water (control), acetic acid (50 ppm) and RF₃ (100 ppm). (C) Worms treated with water (control) and the polysaccharide fractions from different species of medicinal mushrooms of the Orient (100 ppm). Experiments for lifespan measurement were determined at 20° C. Each percent survival (%) was calculated from the ratio of number of living worms to the original number of worms at the indicated time points. Statistical P values were calculated from each group of experiments treated with indicated substances and compared directly with the control group at the same time points. The analysis was done in triplicate and similar results were obtained and analyzed with *P<0.01. The concentrations were represented in ppm (parts per million) based on weigh/volume (w/v) ratios.

FIGS. 2A-D are semi-quantitative reverse transcription polymerase chain reaction (RT-PCR) detection of transcripts for DAF-2, DAF-16, TIR-1, RAB-1, PMK-1 and CLK-1 mRNA in C. elegans on day 2 after treatment. Specific primers for each gene (listed in Table 1) were used in the reaction, and ACT-1 was used as an internal control. Comparison of expression levels of the genes was based on the normalization to the internal control in all RT-PCR data. (A) Expression of DAF-16 after treatments. (Upper panel) C: Control; F: RF₃ (100 ppm); A: Acetic acid (50 ppm); (Lower panel) C: Control; F3: RF₃ (100 ppm); AC: Crude extract of A. camphorate mycelia (100 ppm); HE: Crude extracts of H. erinaceus mycelia (100 ppm); (B) Expression of DAF-2 and TIR-1 after treatments. C: Control; F3: RF₃ (100 ppm); AC: Crude extract of A. camphorate mycelia (100 ppm); HE: Crude extracts of H. erinaceus mycelia (100 ppm); (C) Expression of DAF-16, CLK-1, TIR-1 and RAB-1 and PMK-1 after incubation of C. elegans with (F) or without (N) RF₃ (100 ppm) for 2 days. RAB-1 and PMK-1 are two important genes located in the MAPK pathway. The arrows show elevated transcription levels of the indicated genes after RF₃ treatment, and the horizontal lines indicate no change in mRNA transcription after RF₃ treatment; (D) Time-course analysis of mRNA transcription of TIR-1 and DAF-16 in response to RF₃ stimulation. All experiments were done in triplicate.

FIGS. 3A-B are graphs showing results of Gene knock-out experiments of RNAi on lifespan of C. elegans treated with RF₃. Lifespan measurements were performed using plates containing RNAi bacteria specific for DAF-16 and TIR-1 genes as described in Materials and Methods. (A) Compared with control worms, DAF-16 RNAi shortened the life spans by ˜25% while TIR-1 RNAi extended life spans by ˜18%. (B) RNAi targeting DAF-16 completely prevented lifespan extension by RF₃ treatment. Worms treated with both RF₃ and DAF-16 RNAi had lifespan similar to wild-type control group. On the other hand, TIR-1 RNAi exhibited a weaker effect on lifespan extension of worms treated with RF₃. The worms had shorter life spans than those without knock-out of TIR-1, and still lived much longer than the control group (>18%). The experiments were done in triplicate and results were analyzed with *P<0.01. It is noteworthy that activation of TIR-1 was not likely to be on the exclusive signaling pathway for DAF-16-mediated lifespan extension by RF₃ treatment.

FIGS. 4A-D illustrate the effects of RF₃ and LPS on lifespan regulation and gene expression in C. elegans. (A) Lifespan measurements of worms treated with RF₃ at concentrations of 100 and 500 ppm and LPS at a concentration of 10 ppm. Compared with normal worms (N, control group), lifespan was shortened by ˜13% in worms treated with 10 ppm LPS, and extended by 35% with 100 ppm RF₃. It is to be noted that RF₃ treatment of 500 ppm had a weaker effect on lifespan than that of 100 ppm. (B) Semi-quantitative RT-PCR analysis of tif-1 and DAF-16 mRNA transcription levels treated with 50 ppm acetic acid (A), 100 ppm RF₃ (F) and 10 ppm LPS (L). (C) Real-time q-PCR analysis of TIR-1, DAF-16 and RAB-1 mRNA levels after treatment with 100 ppm RF₃ or 10 ppm LPS, with (white bars) or without (black bars) knock-out of TIR-1 with RNAi (*P value<0.01 analyzed with an unpaired two-tailed t-test; error bars, s.d.). n=4 for both semi-quantitative RT-PCR and real-time q-PCR experiments.

FIG. 5. is a diagram of a scheme for cell-signaling pathways involved in DAF-16-mediated longevity effect in C. elegans treated with acetic acid and polysaccharide fractions from Reishi (RF₃), A. camphorata and H. erinaceus respectively. The up-and-down arrows indicate up- or down-regulation of genes involved in the signaling pathways. It appears that for acetic acid and H. erinaceus the MAPK pathway was apparently not involved. Their longevity-promoting effect was probably mediated by a pathway which reduced DAF-2 expression and indirectly increased the expression of DAF-16 through some unidentified downstream signaling regulation with some unknown factors or cell receptors.

FIG. 6A-B are graphs showing the synergistic effect of acetic acid and RF₃ on the lifespan of C. elegans. (A) Effects of mixtures of RF₃ and acetic acid at different proportions or concentrations. It clearly indicates that various mixtures with different proportions of acetic acid and RF₃ all possess higher activities than either substance used alone. The optimal results were achieved with a mixture containing 50 ppm acetic acid and 100 ppm RF₃. (B) The long-term stability of the mixture of RF₃ (100 ppm) and acetic acid (50 ppm). Even after standing for two weeks, the mixture still maintained the same activity as that of the freshly-prepared RF₃ solution when tested on C. elegans. Tables under the percent-survival curves tabulate the potency of lifespan extension for various mixtures of acetic acid and RF₃ as compared with the control group. All the values shown are means±SD, calculated from triplicate experiments.

FIG. 7 is a table providing specific primers for each gene used in the reaction, identified by SEQ ID NO:1-14, and ACT-1 was used as an internal control as illustrated in FIGS. 2A-D of RT-PCR detection of transcripts for DAF-2, DAF-16, TIR-1, RAB-1, PMK-1 and CLK-1 mRNA in C. elegans on day 2 after treatment.

FIG. 8 is a table providing a list of differentially expressed proteins in C. elegans treated with acetic acid and Reishi polysaccharide fraction RF₃, as shown in FIGS. 9A-D.

FIG. 9A-D illustrates comparative 2-DE gel patterns of C. elegans treated with RF₃ (100 ppm), acetic acid (50 ppm) or a mixture of the two. 150 μg total proteins from lysates of C. elegans were loaded on IPG gel strips (pH 3-10, 13 cm). The IPG strips after IEF were rehydrated and subjected to 2-DE. 2-DE protein profiles of C. elegans were obtained for the control without treatment (A), and treatment with acetic acid (B), RF₃ (C), and their mixture (D). Protein spots marked by No. 1-15 on the maps were found to be differentially expressed; these were further identified by LC-MS/MS and listed in the table of FIG. 8. The 2DE-gel images were scanned using a fluorescence image scanner Typhoon 9400 and analyzed by using PDQuest software. Intensity levels of protein spots in each gel were normalized between gels as a proportion of the total protein intensity detected for the entire gel.

FIG. 10(a)-(d) illustrates (a) a phylogenetic tree calculated from a multiple sequence alignment of the forkhead domains of 16 proteins, the human FOXO proteins FOXO₁, FOXO₃a and FOXO₄, the C. elegans DAF-16 and mouse Foxa₃; (b) dFOXO having three PKB phosphorylation sites in the same orientation as those of mammalian FOXO proteins, with sites indicated above the protein; PEST (destruction), nuclear localization (NLS), nuclear export (NES) and DNA-binding sequences are also shown; (c) a multiple amino-acid sequence alignment of the dFOXO, human FOXO and DAF-16 forkhead domains, with the secondary structure indicated above the alignment and similar and identical amino-acid residues shaded in gray and black, respectively; and (d) the dFOXO gene spanning a genomic region of 31 kilobases (kb) and containing 11 exons (grey bars).

### DETAILED DESCRIPTION

In some exemplary implementations, disclosed is a composition comprising at least one of acetic acid, RF₃ polysaccharide, extract of Antrodia camphorate, and extract of Hericium erinaceus. Such a composition may be administered to regulate DAF-16 expression. It is believed that increased expression of DAF-16 is related to increased longevity.

The composition may be administered to a subject in order to regulate DAF-16 expression. In implementations wherein the composition includes acetic acid, the acetic acid may comprise between about 50 ppm to about 100 ppm of the composition. In implementations wherein the composition includes RF₃ polysaccharide, the RF₃ polysaccharide may comprise between about 100 ppm and about 500 ppm of the composition.

In some exemplary implementations, a composition may contain an extract of RF₃ polysaccharide or an extract of Antrodia camphorate. When administered, a composition containing RF₃ polysaccharide or Antrodia camphorate is believed to act on one or more receptors of a cell to activate the MAPK pathway resulting in increased expression of DAF-16. The MAPK pathway may be activated by expression of TIR-1 and RAB-1, leading to expression of PMK-1, and DAF-16, or by another mechanism leading to expression of RAB-1, PMK-1, and DAF-16. Thus, a composition containing RF₃ polysaccharide or Antrodia camphorate is believed to increase expression of at least TIR-1, RAB-1, PMK-1, and DAF-16.

In some exemplary implementations, a composition may contain acetic acid or an extract of Hericium erinaceus. When administered, a composition containing acetic acid or Hericium erinaceus is believed to act on one or more receptors of a cell to inhibit expression of DAF-2, thereby increasing expression of DAF-16.

At least some of the receptors upon which acetic acid and Hericium erinaceus act is believed to be different than at least some of the receptors on which RF₃ polysaccharide and Antrodia camphorate act. Likewise, separate pathways may be followed to effectuate increased expression of DAF-16, as discussed herein. A composition containing at least one of acetic acid and Hericium erinaceus and at least one of RF₃ polysaccharide and Antrodia camphorate may thereby cause greater expression of DAF-16. For example, a composition comprising at least acetic acid and RF₃ polysaccharide may be administered to regulate DAF-16 expression.

In some exemplary implementations, a method of regulating DAF-16 expression may be employed. The method includes administering a composition comprising at least acetic acid and RF₃ polysaccharide; providing the composition to at least one receptor on a surface of a cell; and causing an increase in expression of DAF-16. The composition may act on a receptor that activates the MAPK pathway by expression of RAB-1, after expression of TIR-1 or by some other mechanism. Activation of the MAPK pathway may include expression of at least PMK-1. The composition may also act on a receptor that inhibits expression of DAF-2, which may lead to the increased expression of DAF-16.

In some exemplary implementations, a method for screening a series of commercial supplements, including some antioxidant vitamins, traditional herb medicines, and vinegars, promoted to be anti-oxidative stress and to boost or enhance immunity to acquire data on if they have an impact the lifespan of C. elegans and the underlying mechanism(s) thereof.

In some exemplary implementation of the present disclosure there is disclosed a compound screening system using C. elegans as a live model organism to examine and evaluate the longevity potential of various compounds. In one aspect a group of natural substances are screened for their effects on the lifespan of that live model.

In some exemplary implementation, a method of screening compounds for their effect on DAF-16 expression in C. elegans the method may comprise administering at least one known compound to C. elegans; comparing the lifespan of C. elegans to which the at least one known compound was administered with the lifespan of control C. elegans to which the at least one known compound was not administered; determining if the lifespan of C. elegans to which the at least one known compound was administered exceeded the normal lifespan by a selected percentage; and, determining if the at least one known compound increased at least DAF-16 expression in C. elegans. At least one known compound may include acetic acid. Determining if the at least one known compound increased at least DAF-16 expression in C. elegans may be accomplished by performing reverse transcription of an isolated and purified RNA sample from the C. elegans to which the at least one known compound was administered. The isolated and purified RNA sample may include at least one of DAF-2, DAF-16, TIR-1, RAB-1 and PMK-1.

According to another aspect, the composition comprising at least one of acetic acid, RF₃ polysaccharide, extract of Antrodia camphorate, and extract of Hericium erinaceus can be included in a pharmaceutical or nutraceutical composition together with additional active agents, carriers, vehicles, excipients, or auxiliary agents identifiable by a person skilled in the art upon reading of the present disclosure.

The pharmaceutical or nutraceutical compositions preferably comprise at least one pharmaceutically acceptable carrier. In such pharmaceutical compositions, the composition comprising at least one of acetic acid, RF₃ polysaccharide, extract of Antrodia camphorate, and extract of Hericium erinaceus forms the “active compound,” also referred to as the “active agent.” As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates, or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Subject as used herein refers to humans and non-human primates (e.g., guerilla, macaque, marmoset), livestock animals (e.g., sheep, cow, horse, donkey, and pig), companion animals (e.g., dog, cat), laboratory test animals (e.g., mouse, rabbit, rat, guinea pig, hamster), captive wild animals (e.g., fox, deer), and any other organisms who can benefit from the agents of the present disclosure. There is no limitation on the type of animal that could benefit from the presently described agents. A subject regardless of whether it is a human or non-human organism may be referred to as a patient, individual, animal, host, or recipient.

Pharmaceutical compositions suitable for an injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition should be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be transmucosal or transdermal. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

According to implementations, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to cell-specific antigens) can also be used as pharmaceutically acceptable carriers. These can be prepared according to methods known to those skilled in the art, for example, as described in U.S. Pat. No. 4,522,811, which is incorporated by reference herein.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects can be used, care should be taken to design a delivery system that targets such compounds to the site of affected location to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of the active compound (i.e., an effective dosage) may range from about 0.001 to 100 g/kg body weight, or other ranges that would be apparent and understood by artisans without undue experimentation. The skilled artisan will appreciate that certain factors can influence the dosage and timing required to effectively treat a subject, including but not limited to the severity of the disease or disorder, previous treatments, the general health or age of the subject, and other diseases present.

According to another aspect, one or more kits of parts can be envisioned by the person skilled in the art, the kits of parts to perform at least one of the methods herein disclosed, the kit of parts comprising two or more compositions, the compositions comprising alone or in combination an effective amount of at least one of acetic acid, RF₃ polysaccharide, extract of Antrodia camphorate, and extract of Hericium erinaceus according to the at least one of the above mentioned methods.

The kits possibly include also compositions comprising active agents other than acetic acid, RF₃ polysaccharide, extract of Antrodia camphorate, and extract of Hericium erinaceus, identifiers of a biological event, or other compounds identifiable by a person skilled upon reading of the present disclosure. The term “identifier” refers to a molecule, metabolite or other compound, such as antibodies, DNA or RNA oligonucleotides, able to discover or determine the existence, presence, or fact of or otherwise detect a biological event under procedures identifiable by a person skilled in the art; exemplary identifiers are antibodies, exemplary procedures are western blot, nitrite assay and RT-PCR, or other procedures as described in the Examples.

The kit can also comprise at least one composition comprising an effective amount at least one of acetic acid, RF₃ polysaccharide, extract of Antrodia camphorate, and extract of Hericium erinaceus or a cell line. The compositions and the cell line of the kits of parts to be used to perform the at least one method herein disclosed according to procedure identifiable by a person skilled in the art.

As used in this application, “DAF-16” means a nucleic acid, nucleic acid product, or protein having at least about 50%, 60%, 70%, 80%, 90%, 95%, 96%, 97%, 98%, 99%, or more, or complete (100%) sequence identity to SEQ ID NO:19. Those skilled in the art will appreciate that the compositions and methods according to implementations of the present disclosure may be effective as they relate to a variety of sequences, including sequences having degrees of homology with SEQ ID NO:19. Moreover, artisans will readily recognize that the term “DAF-16” is known and understood according to other names by artisans, the common feature being substantial homology to SEQ ID NO:19.

As used in this application, “ACT-1”, “CLK-1”, “DAF-2”, “PMK-1”, “RAB-1”, and “TIR-1” mean nucleic acids, nucleic acid products, or proteins corresponding to nucleic acids, nucleic acid products, or proteins known generally to those skilled in the art.

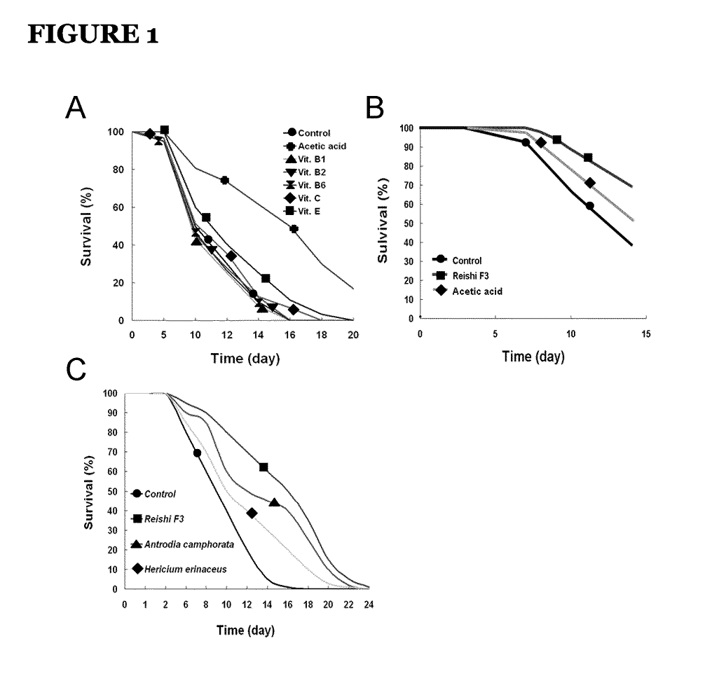

For the testing and screening of natural products alleged to have longevity-promoting properties, wild-type C. elegans (N2) were used as a live model organism. The lifespan of each was measured in the presence or absence of selected natural substances and commercial health-food products. These included vitamins such as vitamins E and C, the vitamin B-complex group, commercial vinegars, and acetic acid, which is one substance found in vinegars, plus some edible mushroom extracts such as RF₃ and mycelium fractions of Antrodia camphorate and Hericium erinaceus (lion's mane mushroom). The lifespan measurements showed that treatment with some of the above-mentioned substances caused a significant extension of lifespan (FIG. 1) when compared with the negative control group (no treatment).

Acetic acid, in contrast to benzoic acid and citric acid concurrently tested on C. elegans showed lifespan-extending effect and have potential for use in higher organisms. The RF₃ polysaccharide fraction, A. camphorata, and H. erinaceus also showed lifespan-extending effect and have potential for use in higher organisms. However, the vitamin group appeared to show no significant longevity effects in C. elegans.

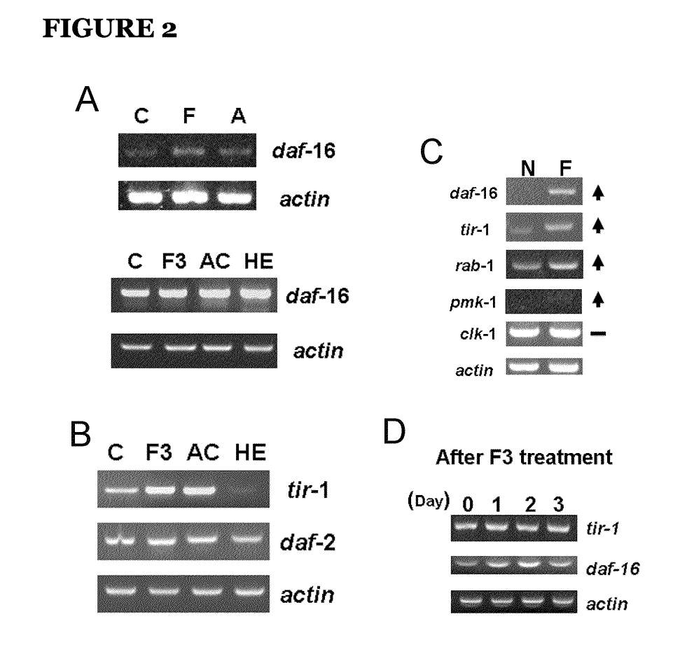

The mechanisms underlying their lifespan-extending effects on C. elegans have been further investigated. A series of experiments by means of reverse transcription-polymerase chain reaction (RT-PCR) were carried out to analyze the transcription profiles of some target genes in C. elegans. These target genes included DAF-2 and DAF-16, which were shown to be involved in pathways that play direct roles in determining lifespan, and the three genes TIR-1, RAB-1 and PMK-1, which are related to Toll-like receptors and MAPK pathway of the host-pathogen immune system. As shown in FIG. 2A, all three mushroom extracts and acetic acid caused significant increase in DAF-16 expression. Previous studies have indicated that increasing DAF-16 could protect cells from oxidative damage by an activation in the expression of antioxidant enzymes such as SOD; thereby resulting in an increased lifespan for C. elegans. Therefore, the findings corroborated that these mushroom extracts could extend lifespan through induction of DAF-16. Although many studies have attributed DAF-16 induction to the decrease or loss of DAF-2 function, only one of the three mushroom extracts tested (H. erinaceus) actually reduced DAF-2 expression. Neither RF₃ nor A. camphorata significantly reduced DAF-2 expression, and yet they both remarkably stimulated TIR-1 expression (FIG. 2B). To understand whether the TIR-1 activation preceded the activation of MAPK pathway, in vivo RAB-1 and PMK-1 transcription levels after RF₃ treatment were further analyzed. The result apparently suggested that after TIR-1-stimulation by RF₃, the MAPK pathway was activated. CLK-1 gene expression, which is another important factor involved in the regulation of DAF-16 expression and supposedly related to a mitochondrial energy-dependent pathway, was also checked. No enhancement of the CLK-1 mRNA signal was observed (FIG. 2C). The time-course study also revealed that the maximal induction of TIR-1 occurred on the first day after RF₃ treatment; whereas the maximal induction level of DAF-16 was observed on the day 2 (FIG. 2D).

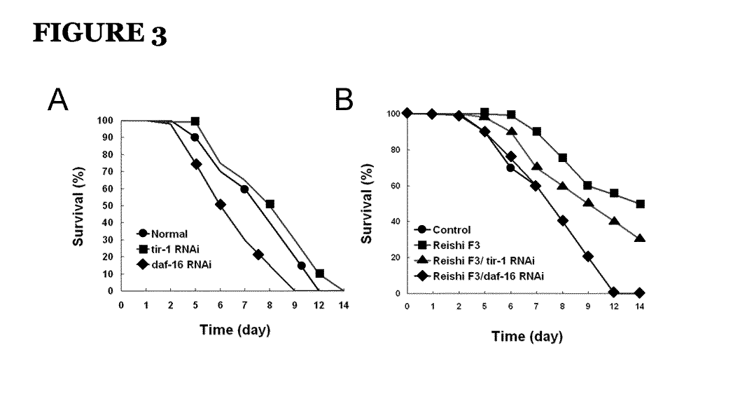

Subsequently, to examine if the increased expression of TIR-1 was the exclusive and direct cause of DAF-16 induction when stimulated by RF₃ polysaccharide, the effect of RF₃ on the lifespan of C. elegans was analyzed by treatment with TIR-1 or DAF-16 RNAi bacteria (FIG. 3). The results showed that the knock-out of DAF-16 expression could completely eliminate the lifespan-extending effect of RF₃, returning C. elegans to its original lifespan. Although the longevity-promoting effect of RF₃ was reduced upon treatment with TIR-1 RNAi, the treated C. elegans still achieved a longer lifespan than untreated worms or those treated with RF₃ and DAF-16 RNAi. This pointed to the fact that the receptors in C. elegans responding to the RF₃ polysaccharide were not limited to the receptors linked to TIR-1; Consequently there are possibly other not-yet-identified receptors associated with the longevity effect of RF₃ reported here.

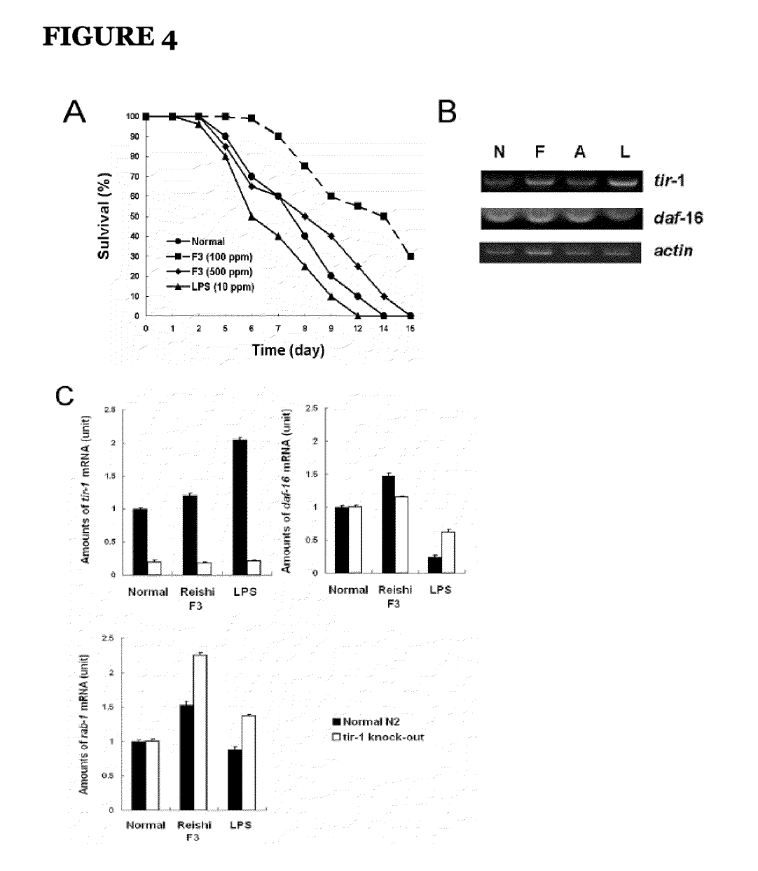

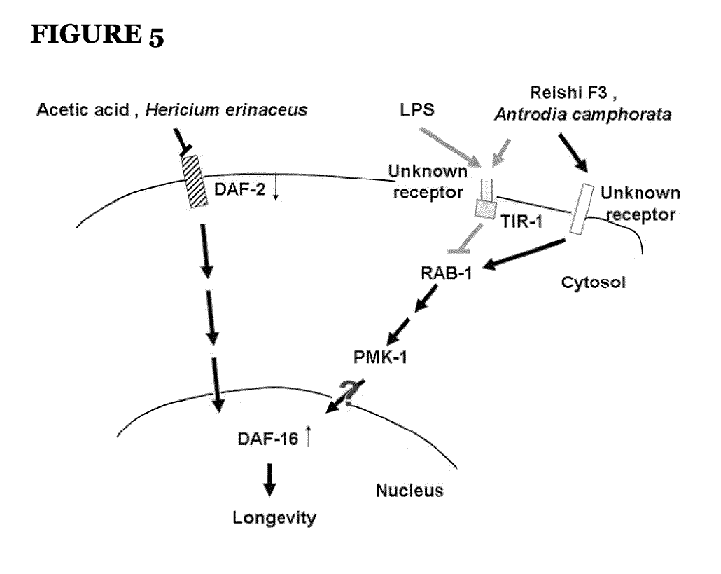

According to some previous reports, RF₃ could stimulate immunomodulation in animals through its binding to the TLR4 receptor, which initiates the same pathway as that of lipopolysaccharide (LPS) endotoxins from bacteria. Therefore, also explored was whether RF₃ polysaccharide and LPS from E. coli have the same effect on TIR-1 mediated MAPK-activation, resulting in DAF-16 expression in C. elegans (FIGS. 4A-C). Feeding worms with RNAi bacteria followed by real-time RT-PCR detection of mRNA levels revealed that both RF₃ and LPS could induce TIR-1 expression to a similar extent (slightly higher with LPS). However, while RF₃ induced DAF-16 expression significantly, the expression was strongly inhibited by LPS under similar conditions (FIG. 4B). Moreover, after knocking out TIR-1, the repression of DAF-16 by LPS was weakened (Right of FIG. 4C). Meanwhile, no matter what treatment was applied, RAB-1 expression always increased when TIR-1 expression was inhibited (Bottom of FIG. 4C). When using A. camphorata under identical conditions to those of RF₃, the same results as with RF₃ were obtained (data not shown). Therefore, it is conceivable that the mechanisms by which RF₃ and A. camphorata promote longevity might be similar. It is therefore suggested that that the polysaccharide fraction RF₃ attaches to at least two different types of receptors on the cell surface. One of the receptors increased TIR-1 expression leading to downstream signal transduction and regulation to reduce the RAB-1 expression. However, they may also bind to another unknown receptor at the same time, which could induce RAB-1 expression followed by activation of the MAPK pathway, resulting in an increase in DAF-16 activity to extend the lifespan of C. elegans. For acetic acid and H. erinaceus, the MAPK pathway was apparently not involved. Their longevity-promoting effect was probably mediated by a pathway which reduced DAF-2 expression and indirectly increased the expression of DAF-16 through some unidentified downstream signaling regulation (FIG. 5).

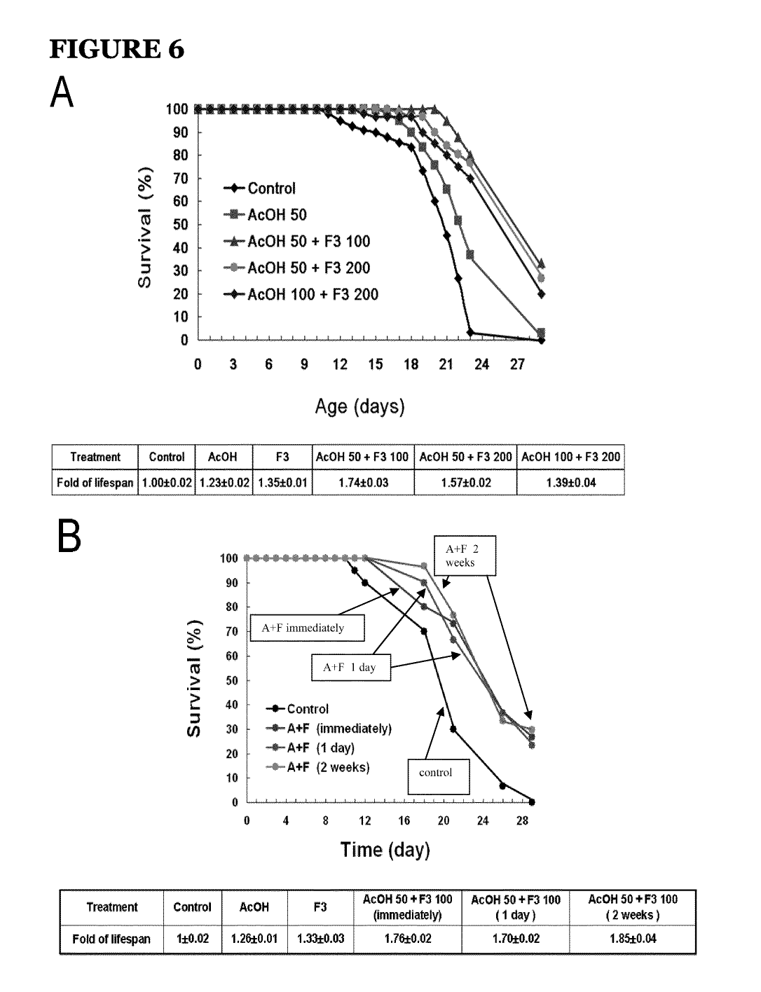

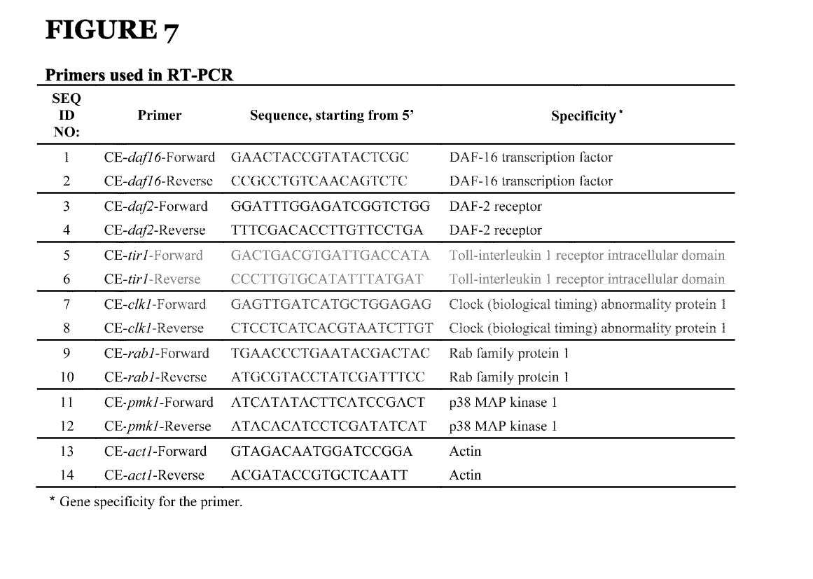

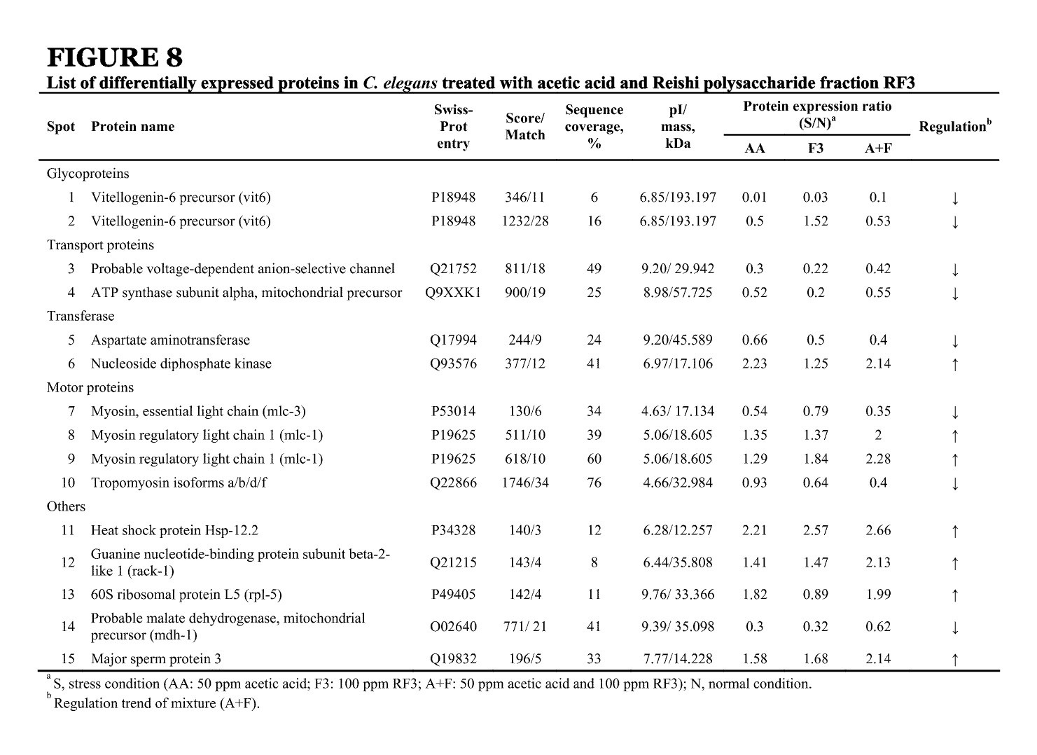

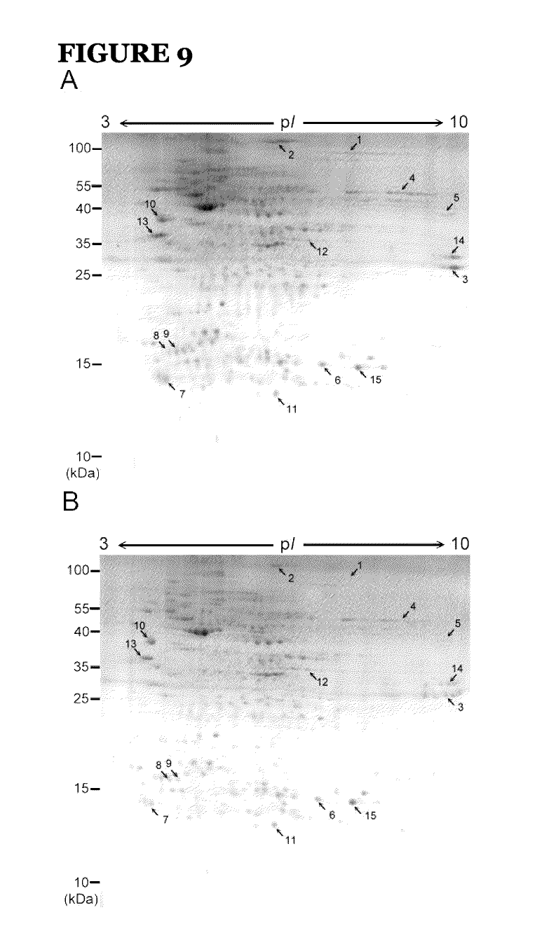

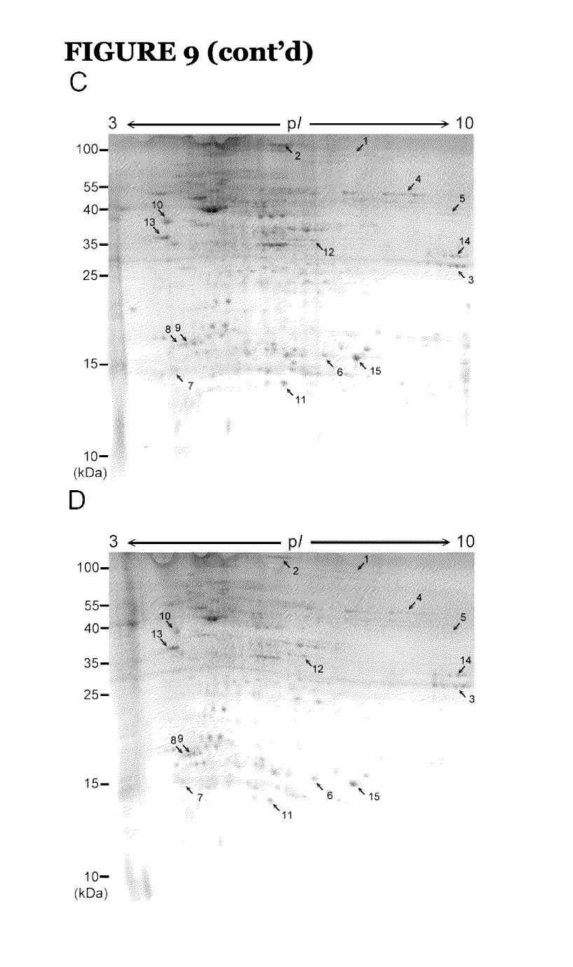

Both acetic acid and RF₃ promoted significantly the lifespan of C. elegans through different mechanisms. Further investigated was whether there was a combinatory or synergistic effect when these two substances were combined and applied to the organism. It is of interest to find that various mixtures with different proportions of acetic acid and RF₃ all possess higher activities than either substance used alone (FIG. 6A). Among these, a solution of 50 ppm (w/v) acetic acid and 100 ppm RF₃ showed the highest activity, achieving a lifespan extension 1.4 times that of 50 ppm acetic acid and 1.3 times that of 100 ppm RF₃ used alone.

To test one aspect of this mixture as a supplement, an active compound, or an active compound to regulate expression of at least DAF-16, the long-term stability of RF₃ in 5% acetic acid (about the minimum acidity contained in vinegar products of US) was also tested. Even after standing for two weeks, the mixture still maintained the same activity as that of the freshly-prepared RF₃ solution when tested on C. elegans (FIG. 6B).

Among tested substances, acetic acid and Reishi polysaccharide RF₃ were shown to generate dramatic positive effects, an increase in lifespan coupled with elevated gene expression of DAF-16, a lifespan-related transcription factor. The mechanisms underlying the lifespan-extension signaling pathways can be identified and validated by employing RNAi coupled with RT-PCR and real-time qPCR. Thus by observing the lifecycle of this experimental organism in the presence of some natural substances or synthetic compounds, it is feasible to achieve the goal of extending lifespan by selecting the right combination of simple compounds like acetic acid and the active ingredient polysaccharide RF₃ from Reishi mushroom demonstrated herein. The study also fulfills an urgent need to develop an efficient and inexpensive in vivo system for evaluating the medicinal functions of natural substances.

### Example 1

Strains, bacteria, natural products and chemicals. The wild-type N2 strain of C. elegans were used in all experiments. The worms were maintained and cultured on nematode growth medium (NGM) agar plates or in liquid medium with E. coli OP₅₀ as a food source. Worms were harvested and washed with Mg buffer (22 mM KH₂PO₄, 42 mM Na₂HPO₄, 86 mM NaCl and 1 mM MgSO₄). Contaminating food and worm debris were removed by sucrose floatation. In addition to OP₅₀, two E. coli strains for RNAi were used (III-1N20 for TIR-1 RNAi and I-5M₂₄ for DAF-16 RNAi). RF₃, crude mycelium-powders of A. camphorate and H. erinaceus were prepared as described in previous papers. Each solution was diluted with deionized water to make test solutions of desired concentrations for various assays.

### Example 2

Lifespan analysis after treatment with various substances. Lifespan assays were performed in 12-well tissue culture plates. Each well contained 1-2 ml of NGM medium, supplemented with 1 mg/ml erythromycin to prevent bacterial division, and 100 μl solution of test substance at the desired concentration. The analyses after treatments were either performed at L₄-larvae or adult stage only. Before treatments, 100 μM of 2 fluoro-5 deoxyuridine (FUDR, Sigma, St. Louis, Mo., USA) was added to the culture medium to prevent any progeny of the test subjects from developing into adults. Briefly, worms were grown to the L₄/young stage on NGM plates seeded with OP₅₀ bacteria, then at least 60 worms were transferred into each of three wells for triplicate assays. All the plates were maintained at 20° C., as described previously, with gentle gyratory shaking. The wells were scored for live and dead worms at appropriate time intervals. A worm was considered dead when it failed to respond to plate tapping or a gentle touch with a platinum wire when observed by using a microscope (Olympus 1×71, NY, USA). The ANOVA program was used for statistical analysis and to determine means and percentiles. In all assays, P values were calculated using the log-rank (Mantel-Cox) method.

### Example 3

RNA preparation and reverse transcriptase-PCR(RT-PCR). Total RNA from batches of C. elegans were isolated from samples in the NGM medium at appropriate time intervals using an RNeasy mini kit (Qiagen). Subsequently, RNA (500 ng) from each sample was reverse-transcribed and PCR was performed using a Superscript One-step RT-PCR kit (Invitrogen). One PCR cycle consisted of the following steps: 1.94° C. for 15 s, 50-56° C. for 30 s and 72° C. for 45 s; 2. repeating for 40 cycles; 3. a final extension of 7 minutes at 72° C. Primer sequences were designed as listed in Table 1. PCR products were run on 1.8% agarose gel and stained with 0.4 μg/ml ethidium bromide. Stained bands were visualized under UV light and photographed with a camera (Nikon E4500).

### Example 4

Quantitative reverse transcriptase-polymerase chain reaction (qRT-PCR) analysis. For each experiment, approximately 100 L₄-larvae or young adult worms were assayed. Total RNA was isolated from worms incubated at 20° C. for the purpose of measuring TIR-1 and RAB-1 mRNA levels on day 1 after treatment. However, if the total RNA was being tested for DAF-16 mRNA level, the worms were harvested on day 2. Isolation, purification, and reverse transcription of C. elegans total RNA were carried out as described before. Quantitative reverse transcriptase-polymerase chain reactions (qRT-PCR) were performed in an RG-3000 real-time PCR System (Corbett Research, Australia) and analyzed using Rotor-Gene Real-time analysis software 6.1 (Corbett Research); mRNA levels of ACT-1 were used for normalization. Primer sequences are available upon request.

### Example 5

RNA interference (RNAi) analysis. In the whole-life RNAi analysis, eggs were added to agar plates seeded with the gene-specific RNAi bacteria (E. coli strain HT115). In drug-treatment experiments, eggs were added to plates seeded with OP₅₀ and grow to the stage of L₄ worms, then transferred to fresh liquid NGM medium that contained gene-specific RNAi bacteria. 100 μM of 2 fluoro-5 deoxyuridine (FUDR) was also added to the medium to prevent any progeny of the test subjects from developing into adults. After two days, the worms were fed with substances being tested. All the lifespan measurements of animals with RNAi were scored as above. In all experiments, the pre-fertile period of adulthood was used as t=o for lifespan analysis. ANOVA was also used for statistical analysis and to determine means and percentiles. In all assays, P values were calculated using the log-rank (Mantel-Cox) method.

### Example 6

Green Fluorescent Protein (GFP)-expression Assays. The transgenic worms carrying DAF-16::gfp transcriptional reporter construct were used for GFP-expression assays. To test whether the expression of GFP exemplified specifically the expression of DAF-16, we used DAF-16 and TIR-1 RNAi to determine its specificity. Fluorescent images of the worms were taken at appropriate time intervals depending upon the lifespan analysis after treatments with RF₃ and/or acetic acid under a fluorescent microscope (Olympus 1×71, NY, USA) and the affiliated digital camera (Olympus DP controller). All the experiments were repeated in duplicate with consistent and similar image results.

### Example 7

2-DE and Image Analysis. L₄ larvae or young adult worms were transferred to freshly-prepared liquid NGM medium and cultured at 20° C. for 4 days. 100 μM FUDR, 50 ppm acetic acid, and 100 ppm RF₃ were then added to the medium. After 3-day treatment, the worms were solubilized in lysis buffer containing 8 M urea, 0.5% Triton X-100 and protease inhibitor cocktail, frozen in liquid nitrogen, and then pulverized into fine powders with a mortar. The homogenates were sonicated and the supernatants after centrifugation were collected and used as protein-loading samples. 150 μg total protein as estimated by protein-content determination using 2-D Quant Kit (Amersham Biosciences), was loaded onto immobilized pH gradient (IPG) gel strips (pH 3-10, 13 cm, Amersham Biosciences). The IPG strips were rehydrated overnight. For the first-dimensional separation, IEF was carried out using Ettan IPGphor II (Amersham Biosciences) at 300-8000 V for 16 h. After IEF, the IPG strips were equilibrated for 10 min each in two equilibration solutions (50 mM Tris-HCl, pH 8.8, 6 M urea, 2% SDS, 30% glycerol containing 100 mg dithiothreitol (DTT) or 250 mg iodoacetic acid, respectively), and the second-dimensional electrophoresis was conducted at 130-250 V for 5-6 h. The gels were stained by Sypro-Ruby overnight. The 2-DE gel images were scanned using a fluorescence image scanner Typhoon 9400 (Amersham Biosciences) and analyzed by using PDQuest software (Bio-Rad). Intensity levels were normalized between gels as a proportion of the total protein intensity detected for the entire gel.

### Example 8

In-gel Digestion and LC-MS/MS. Based on the 2-DE analysis of samples under different treatments, we selected 15 differentially expressed proteins (based on at least 2-fold protein-expression change between control and treated samples) for further identification by LC-MS/MS (nano ESI-Q/TOF) at the proteomic core facility of the Institute of Biological Chemistry, Academia Sinica. The protein spots were cut from 2-D gels, and then destained three times with 25 mM ammonium bicarbonate buffer (pH 8.0) in 50% acetonitrile (ACN) for 1 h. The gel pieces were dehydrated in 100% ACN for 5 min and then dried for 30 min in a vacuum centrifuge. Enzyme digestion was performed by adding 0.5 μg trypsin in 25 mM ammonium bicarbonate per sample at 37° C. for 16 h. The peptide fragments were extracted twice with 50 μl 50% ACN/0.1% TFA. After removal of ACN and TFA by centrifugation in a vacuum centrifuge, samples were dissolved in 0.1% formic acid/50% ACN and analyzed by LC-nanoESI-MS/MS. Proteins were identified in NCBI databases based on MS/MS ion search with the MASCOT program.

### Example 9

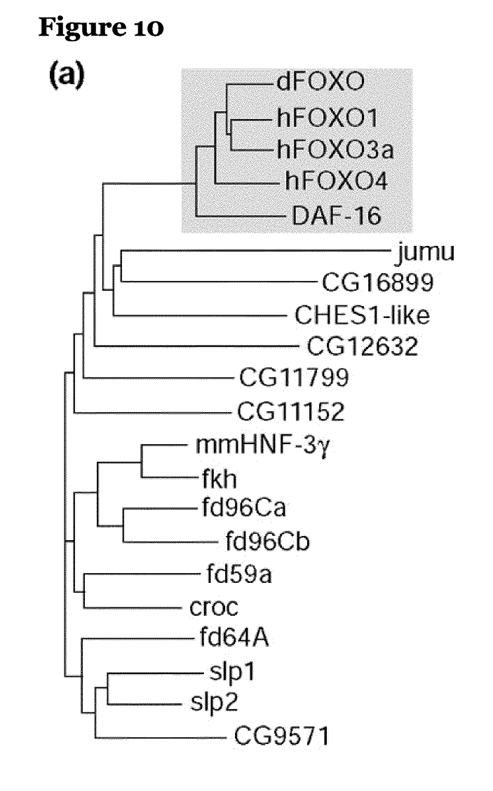

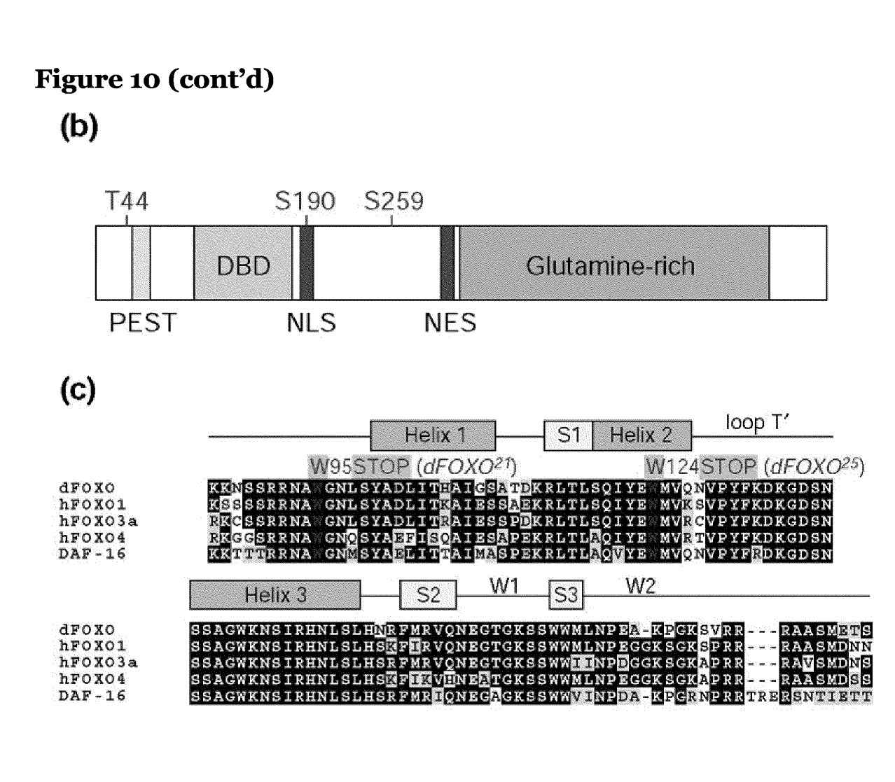

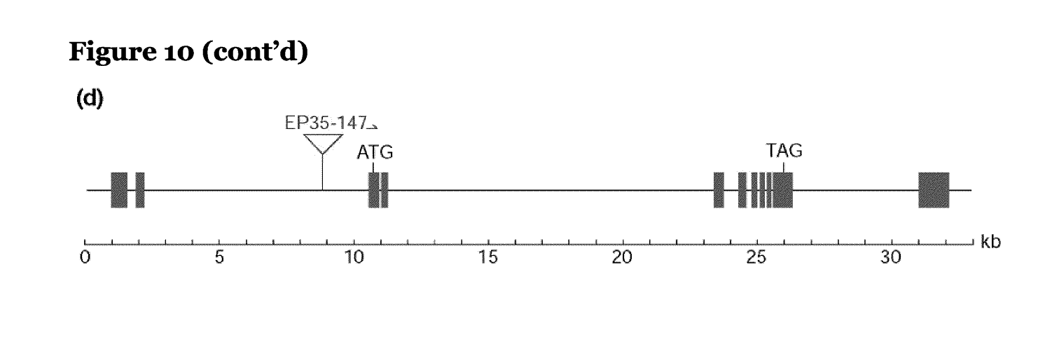

With reference to FIG. 10, the human homologue of DAF-16 shares considerable homology with DAF-16 (C. elegans) and a high degree of conservation in critical binding regions, especially within the DNA-binding domain (see FIG. 10(c)). In humans, FOXO functions as transcription factors to modulate the expression of genes involved in apoptosis, cell cycle, DNA damage repair, oxidative stress, cell differentiation, glucose metabolism and other cellular functions in the same way that DAF-16 functions in C. elegans. Thus, use of acetic acid, RF₃, or RF₃ plus acetic acid in combination, modulates the longevity of humans.

The following publications are hereby incorporated by reference, as if fully set forth herein: A. salminena, J. ojala, J. huuskonen, A. Kauppinena, T. Suuronen, K. kaarnirantac, Interaction of aging-associated signaling cascades: Inhibition of NF-kB signaling by longevity factors FoxOs and SIRT₁, Cell. Mol. Life. Sci. 65 (2008) 1049-1058; Haojie Huang, Donald J. Tindall, Dynamic FoxO transcription factors, J. of Cell Science 120 (2007), 2479-2487; and Coleen T. Murphy, The search for DAF-16/FOXO transcriptional targets: Approaches and discoveries, Experimental Gerontology 41 (2006), 910-921.

With reference to FIG. 10(c), a multiple amino-acid sequence alignment of SEQ ID NO:15 (dFOXO); SEQ ID NO:16 (hFOXO₁); SEQ ID NO:17 (hFOXO₃a); SEQ ID NO:18 (hFOXO₄); and SEQ ID NO:19 (DAF-16) illustrates the homology of the human, C. elegans, and drosophilia versions of DAF-16/FOXO. Amino-acid sequences shown correspond to the DNA binding domains of the respective proteins. The dFOXO (Drosophila), hFOXO (human), and DAF-16 (C. elegans) forkhead domains illustrate the high degree of sequence conservation especially within the DNA-binding domain. The secondary structure is indicated above the alignment. Similar and identical amino-acid residues are shaded in gray and black, respectively. The region encoding helix 3 of the forkhead domain, which is the DNA-recognition helix contacting the major groove of the DNA double helix, is identical in the five proteins. Therefore, each of these proteins contacts insulin response elements through helix 3.

While the apparatus and method have been described in terms of what are presently considered to be the most practical and preferred implementations, it is to be understood that the disclosure need not be limited to the disclosed implementations. It is intended to cover various modifications and similar arrangements included within the spirit and scope of the claims, the scope of which should be accorded the broadest interpretation so as to encompass all such modifications and similar structures. The present disclosure includes any and all implementations of the following claims.

1. A composition, comprising, in combination:
at least acetic acid and an RF₃ polysaccharide;
wherein the at least acetic acid and the RF₃ polysaccharide is effective to increase expression of DAF-16 having a sequence corresponding to SEQ ID NO:19.

2. The composition of claim 1, wherein the composition comprises between about 50 ppm to about 100 ppm acetic acid.

3. The composition of claim 1, wherein the composition comprises between about 100 ppm and about 500 ppm RF₃ polysaccharide.

4. The composition of claim 1, wherein the composition comprises about 50 ppm acetic acid and about 100 ppm RF₃ polysaccharide.

5. The composition of claim 1, further comprising at least one of an extract of Antrodia camphorate and an extract of Hericium erinaceus.

6. The composition of claim 1, wherein the at least acetic acid and the RF₃ polysaccharide is effective to increase the life-span of C. elegans

7. The composition of claim 1, wherein the at least acetic acid and the RF₃ polysaccharide is effective to increase the life-span of human

8. A composition, comprising, in combination:
at least acetic acid and an RF₃ polysaccharide;
wherein the at least acetic acid and the RF₃ polysaccharide increases expression of a protein which possesses an amino-acid sequence with at least 70% sequence homology to SEQ ID NO:19.

9. A method of regulating DAF-16 expression, comprising:
administering a composition comprising at least acetic acid and an RF₃ polysaccharide;
providing the composition to at least one receptor on a surface of a cell;
causing an increase in expression of DAF-16 having a sequence corresponding to SEQ ID NO:19.

10. The method of claim 6, wherein the at least one receptor includes TLR4.

11. The method of claim 6, wherein the causing an increase in expression of DAF-16 includes activating the MAPK pathway.

12. The method of claim 6, wherein the causing an increase in expression of DAF-16 includes causing an increase in expression of RAB-1.

13. The method of claim 6, wherein the causing an increase in expression of DAF-16 includes causing an increase in expression of PMK-1.

14. The method of claim 6, wherein the causing an increase in expression of DAF-16 includes inhibiting expression of DAF-2.

15. A method of screening compounds for their effect on DAF-16 expression in C. elegans the method, comprising:
administering at least one known compound to C. elegans;
comparing the lifespan of C. elegans to which the at least one known compound was administered with the lifespan of control C. elegans to which the at least one known compound was not administered;
determining if the lifespan of C. elegans to which the at least one known compound was administered exceeded the normal lifespan by a selected percentage; and,
determining if the at least one known compound increased at least DAF-16 expression in C. elegans.

16. The method of claim 13, wherein at least one known compound includes acetic acid.

17. The method of claim 13, wherein determining if the at least one known compound increased at least DAF-16 expression in C. elegans is accomplished by performing reverse transcription of an isolated and purified RNA sample from the C. elegans to which the at least one known compound was administered.

18. The method of claim 13, wherein the isolated and purified RNA sample includes at least one of DAF-2, DAF-16, TIR-1, RAB-1 and PMK-1.

19. A composition, comprising, in combination:
at least acetic acid;
wherein the at least acetic acid is effective to increase expression of DAF-16 having a sequence corresponding to SEQ ID NO:19.

20. A composition, comprising, in combination:
at least an RF₃ polysaccharide;
wherein the at least the RF₃ polysaccharide is effective to increase expression of DAF-16 having a sequence corresponding to SEQ ID NO:19.


SEQUENCE LISTING (19 sequences)

SEQ ID NO: 1 - DNA, 18 bp, Artificial Sequence
  Primer
gaactaccgtatactcgc

SEQ ID NO: 2 - DNA, 18 bp, Artificial Sequence
  Primer
ccgcctgtcaacagtctc

SEQ ID NO: 3 - DNA, 20 bp, Artificial Sequence
  Primer
ggatttggagatcggtctgg

SEQ ID NO: 4 - DNA, 20 bp, Artificial Sequence
  Primer
tttcgacaccttgttcctga

SEQ ID NO: 5 - DNA, 20 bp, Artificial Sequence
  Primer
gactgacgtgattgaccata

SEQ ID NO: 6 - DNA, 20 bp, Artificial Sequence
  Primer
cccttgtgcatatttatgat

SEQ ID NO: 7 - DNA, 20 bp, Artificial Sequence
  Primer
gagttgatcatgctggagag

SEQ ID NO: 8 - DNA, 21 bp, Artificial Sequence
  Primer
ctcctcatcacgtaatcttgt

SEQ ID NO: 9 - DNA, 20 bp, Artificial Sequence
  Primer
tgaaccctgaatacgactac

SEQ ID NO: 10 - DNA, 20 bp, Artificial Sequence
  Primer
atgcgtacctatcgatttcc

SEQ ID NO: 11 - DNA, 20 bp, Artificial Sequence
  Primer
atcatatacttcatccgact

SEQ ID NO: 12 - DNA, 20 bp, Artificial Sequence
  Primer
atacacatcctcgatatcat

SEQ ID NO: 13 - DNA, 18 bp, Artificial Sequence
  Primer
gtagacaatggatccgga

SEQ ID NO: 14 - DNA, 18 bp, Artificial Sequence
  Primer
acgataccgtgctcaatt

SEQ ID NO: 15 - protein, 109 aa, Drosophila melanogaster
KKNSSRRNAWGNLSYADLITHAIGSATDKRLTLSQIYEWMVQNVPYFKDKGDSNSSAGWKNSIRHNLSLHNRFMRVQNEGTGKSSWWMLNPEAKPGKSVRRRAASMETS

SEQ ID NO: 16 - protein, 110 aa, Homo sapiens
KSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNN

SEQ ID NO: 17 - protein, 110 aa, Homo sapiens
RKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNS

SEQ ID NO: 18 - protein, 110 aa, Homo sapiens
RKGGSRRNAWGNQSYAEFISQAIESAPEKRLTLAQIYEWMVRTVPYFKDKGDSNSSAGWKNSIRHNLSLHSKFIKVHNEATGKSSWWMLNPEGGKSGKAPRRRAASMDSS

SEQ ID NO: 19 - protein, 112 aa, Caenorhabditis elegans
KKTTTRRNAWGNMSYAELITTAIMASPEKRLTLAQVYEWMVQNVPYFRDKGDSNSSAGWKNSIRHNLSLHSRFMRIQNEGAGKSSWWVINPDAKPGRNPRRTRERSNTIETT